(19) United States
(12) Patent Application Publication
(10) Pub. No.: US 2025/0332269 A1
(43) Pub. Date: Oct. 30, 2025

(54) NON-IMMUNOGENIC, HIGH DENSITY POEGMA CONJUGATES

(71) Applicant: Duke University, Durham, NC (US)

(72) Inventors: Ashutosh Chilkoti, Durham, NC (US); Imran Ozer, Durham, NC (US); Angus Hucknall, Durham, NC (US)

(21) Appl. No.: 18/683,650

(22) PCT Filed: Aug. 23, 2022
(86) PCT No.: PCT/US2022/041251

§ 371 Date: Feb. 14, 2024

Related U.S. Application Data
(60) Provisional application No. 63/236,064, filed on Aug. 23, 2021.

(51) Int. Cl. A61K 47/60; A61K 38/44

(52) U.S. Cl. CPC A61K 47/60; A61K 38/44

(57) ABSTRACT

Disclosed are high density POEGMA-biologically active agent conjugates that have advantageous pharmacokinetics, while also having a reduced or eliminated host-immune response. An example conjugate includes a biologically active agent and a plurality of POEGMA molecules conjugated to the biologically active agent, each POEGMA molecule having a poly(methyl methacry late) backbone and a plurality of side chains covalently attached to the backbone, each side chain including 2 to 9 monomers of ethylene glycol repeated in tandem. Also disclosed are methods of reducing the immunogenicity of a polymer-biologically active agent conjugate.

### CROSS-REFERENCE TO RELATED APPLICATIONS

This application claims priority to U.S. Provisional Patent Application No. 63/236,064 filed on Aug. 23, 2021, which is incorporated fully herein by reference.

### STATEMENT REGARDING FEDERALLY SPONSORED RESEARCH

This invention was made with government support under grant number R41 TR003255-01 awarded by the National Institutes of Health. The government has certain rights in the invention.

### TECHNICAL FIELD

This disclosure relates to biologically active agent-poly[oligo(ethylene glycol) ether methacrylate] (POEGMA) conjugates.

### INTRODUCTION

PEGylation of biologically active molecules, such as proteins and drugs, have found widespread use in biotechnology. However, this extensive use can lead to deleterious immune system consequences. As an example, unmodified uricase is limited as a drug because of its small size, high immunogenicity, and low solubility at physiological pH. Even though PEGylated uricase can mitigate some of these problems, its long-term utility has remained limited in treating diseases, such as chronic refractory gout. For example, in clinical trials, pegloticase (having ˜32 polyethylene glycol (PEG) chains on average per uricase tetramer) induced a significant PEG-specific immune response in 91% of the patients after administering the first dose of treatment, resulting in high-titers of anti-drug antibodies (ADA) in patients that accelerated the clearance of the drug and resulted in a treatment response rate of only 20-49%. In addition, ˜50% of the patients with high titers of PEG antibodies experienced infusion reactions —26% being severe and 6.5% characterized as life-threatening anaphylaxis—upon administration of subsequent doses due to activation of the complement system by the induced PEG antibodies. Further, pegloticase resulted in severe infusion reactions with high PEG-specific pre-existing IgG titers. Together, these clinical problems resulted in the withdrawal of the drug from the European market and limited its used elsewhere.

### SUMMARY

In one aspect, provided are conjugates including a biologically active agent; and a plurality of POEGMA molecules conjugated to the biologically active agent, each POEGMA molecule having a poly(methyl methacrylate) backbone and a plurality of side chains covalently attached to the backbone, each side chain including 2 to 9 monomers of ethylene glycol (EG) repeated in tandem, wherein the conjugate includes about 5 to about 130 POEGMA molecules per biologically active agent.

In another aspect, provided are methods of reducing the immunogenicity of a polymer-biologically active agent conjugate, the method including: conjugating about 5 to about 130 POEGMA molecules to a biologically active agent, each POEGMA molecule having a poly(methyl methacrylate) backbone and a plurality of side chains covalently attached to the backbone, each side chain including 2 to 9 monomers of EG repeated in tandem to provide a conjugate, wherein the conjugate has a reduced immune response relative to a PEG-biologically active agent conjugate including about 5 to about 130 PEG molecules per biologically active agent.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 is a schematic overview of example uricase-POEGMA conjugate synthesis. (A) Recombinant expression and purification of uricase-t-ELP (UTE). (B) Tobacco etch virus (TEV) protease-mediated cleavage and purification by inverse transition cycling (ITC) to generate uricase tetramers. (C) activators regenerated by electron transfer atom transfer radical polymerization (ARGET-ATRP) of OH-functional POEGMA using EG3 monomers, followed by chain-end nitrophenyl carbonate (NPC) activation. (D) Conjugation of NPC-POEGMA to uricase tetramer, yielding example uricase-POEGMA conjugates.

FIG. 2 shows synthesis and characterization of example uricase conjugates. FIG. 2A is a Coomassie-stained sodium dodecyl sulfate-polyacrylamide gel electrophoresis (SDS-PAGE) analysis of TEV-protease mediated UTE cleavage. L1: Ladder; L2: (i) undissociated trimeric UTE, possibly due to ELP preventing complete disassociation and (ii) pure monomeric; L3: (iii) His₆-ELP-TEV and (iv) its truncation product His₆-ELP; L4: Reaction mix at t=0; L5: Reaction mix t=20 h showing (iv) free uricase monomers and (v) cleaved ELP; L6: ITC-purified uricase monomer. FIG. 2B is a series of size exclusion chromatography (SEC) traces of uricase variants. Peaks eluting at 18-19 min and 19-21 min correspond to native octameric (0.8 mass %) and tetrameric (99.2 mass %) uricase, respectively, measured by multi-angle light scattering. FIG. 2C is dynamic light scattering (DLS) analysis of uricase variants.

FIG. 3 shows pharmacokinetics (PK) analysis of example uricase conjugates. Plasma concentrations of sterile, endotoxin-free, and fluorescently labeled FIG. 3A unmodified uricase and FIG. 3B uricase conjugates in C57BL/6J (n=5) mice after a single i.v. administration at a dose of 36.6 nmol kg⁻¹. Plasma concentration of the drugs was tracked by collecting blood at pre-determined time points for 144 hours, followed by processing them into plasma and measuring fluorescence using a plate reader. Data showed the mean±standard error of the mean (SEM). Data were fitted to a one-phase exponential decay curve using GraphPad Prism 9.

FIG. 4 shows ADA response induced by different treatments. FIG. 4A is a schematic of the treatment and blood collection regimen (n=10 mice per group). ADA response was measured for each mouse using a validated Luminex multiplexed assay (n=6 per plasma sample). IgM response on (FIG. 4B) Day 10 and (FIG. 4C) Day 44. IgG response on (FIG. 4D) Day 10 and (FIG. 4E) Day 44. OVA-PEG-and OVA-POEGMA-coupled beads show the PEG-specific and POEGMA-specific immune response in plasma samples of mice treated with phosphate-buffered saline (PBS), uricase-PEG_{Mw}, and uricase-POEGMA. Data were normalized to the signal measured with mouse IgG- and IgM-coupled beads (positive controls) and represented as the average ADA response in a treatment group±SEM. Data were analyzed by two-way repeated-measures ANOVA, followed by post-hoc Tukey's multiple comparison test. A test was considered statistically significant when p<0.05. ****p<0.0001. Not significant (ns).

FIG. 5 shows PEG antibodies do not have reactivity to example uricase-POEGMA conjugates. The reactivity of PEG antibodies to uricase, uricase-PEG_{Mw}, and uricase-POEGMA was tested using (FIG. 5A) indirect and (FIG. 5B) competitive ELISA. In indirect ELISA, the antigens were absorbed on the well-plate (n=4) and reacted with the polyclonal PEG antibodies present in OVA-PEG-immunized mice plasma. In the competitive ELISA, exendin-PEG was absorbed onto the well-plate. The antigens (uricase, uricase-PEG_{Mw}, and uricase-POEGMA) (n=4) pre-mixed OVA-PEG-treated mice plasma competed with exendin-PEG for binding to the PEG antibodies present. Data represented the mean absorbance±SEM and were analyzed using two-way ANOVA, followed by post-hoc Tukey's multiple comparison test. A test was considered statistically significant when p<0.05. *p<0.05, **p<0.01, ***p<0.001, and ****p<0.0001. Not significant (ns).

### DETAILED DESCRIPTION

Placing a repetitive, high density arrangement of a potential antigen on the surface of a highly immunogenic molecule, such as a protein, may alter epitope exposure to antibodies, as well as potentially induce a heightened immune response led by both IgM and IgG class antibodies. At the time of filing the present application, it was not known if presenting POEGMA at high densities on a biologically active agent would lead to repetition-based activation of the immune system, which could alter recognition to PEG antibodies and potentially engender an immune response to POEGMA itself.

The present disclosure found that even at high densities, POEGMA conjugated uricase did not bind PEG antibodies and remained non-immunogenic. This is in contrast to a high density PEG-uricase counterpart that not only reacted with PEG antibodies, but also induced both an IgM and IgG antibody response-likely due to the high density of PEG epitopes on the surface of uricase. Not only did the disclosed high density POEGMA-uricase conjugates avoid the immune-based drawbacks of its PEG-based counterpart, but the disclosed conjugates also significantly outperformed the pharmacokinetic profile of these counterparts. Accordingly, the disclosed conjugates provide better PK benefits compared to similar PEG-based systems, while also avoiding the immune system pitfalls associated with these same PEG-based systems. Ultimately, the findings of the present disclosure can potentially solve problems limiting the clinical utility of biologically active agents, such as uricase, in treating diseases.

### 1. Definitions

Unless otherwise defined, all technical and scientific terms used herein have the same meaning as commonly understood by one of ordinary skill in the art. In case of conflict, the present document, including definitions, will control. The materials, methods, and examples disclosed herein are illustrative only and not intended to be limiting. Methods and materials similar or equivalent to those described herein can be used in practice or testing of the disclosed invention. All publications, patent applications, patents and other references mentioned herein are incorporated by reference in their entirety.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The term “alkyl” refers to a straight or branched, saturated hydrocarbon chain containing from 1 to 10 carbon atoms. The term “C₁-C₄ alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, and n-hexyl.

The term “amide” refers to the group —C(O)NR wherein R is selected from the group consisting of hydrogen, alkyl, alkenyl, and alkynyl, any of which may be optionally substituted, e.g., with one or more substituents.

The term “antigen” refers to a molecule capable of being bound by an antibody or a T cell receptor. The term “antigen” also encompasses T-cell epitopes. An antigen is additionally capable of being recognized by the immune system and/or being capable of inducing a humoral immune response and/or cellular immune response leading to the activation of B-lymphocytes and/or T-lymphocytes. In some embodiments, the antigen contains or is linked to a Th cell epitope. An antigen can have one or more epitopes (B-epitopes and T-epitopes). Antigens may include polypeptides, polynucleotides, carbohydrates, lipids, small molecules, polymers, polymer conjugates, and combinations thereof. Antigens may also be mixtures of several individual antigens.

The term “antigenicity” refers to the ability of an antigen to specifically bind to a T cell receptor or antibody and includes the reactivity of an antigen toward pre-existing antibodies in a subject.

The term “biologically active agent” refers to a substance that can act on a cell, virus, tissue, organ, organism, or the like, to create a change in the functioning of the cell, virus, tissue, organ, or organism. Examples of a biologically active agent include, but are not limited to, small molecule drugs, lipids, proteins, peptides, and nucleic acids. A biologically active agent is capable of treating and/or ameliorating a condition or disease, or one or more symptoms thereof, in a subject. Biologically active agents of the present disclosure also include prodrug forms of the agent.

The term “carboxyl” refers to the group —C(═O)OR, wherein R is selected from the group consisting of hydrogen, alkyl, alkenyl, and alkynyl, any of which may be optionally substituted, e.g., with one or more substituents.

The term “effective amount” or “therapeutically effective amount” refers to an amount sufficient to effect beneficial or desirable biological and/or clinical results.

The term “ester” refers to the group —C(O)OR wherein R is selected from the group consisting of hydrogen, alkyl, alkenyl, and alkynyl, any of which may be optionally substituted, e.g., with one or more substituents.

The term “hydroxyl” or “hydroxy” refers to an —OH group.

The term “immunogenicity” refers to the ability of an antigen to induce an immune response and includes the intrinsic ability of an antigen to generate antibodies in a subject. As used herein, the terms “antigenicity” and “immunogenicity” refer to different aspects of the immune system and are not interchangeable.

The term “subject” includes humans and mammals (e.g., mice, rats, pigs, cats, dogs, and horses). Typical subjects of the present disclosure may include mammals, particularly primates, and especially humans. For veterinary applications, suitable subjects may include, for example, livestock such as cattle, sheep, goats, cows, swine, and the like; poultry such as chickens, ducks, geese, turkeys, and the like, as well as domesticated animals particularly pets such as dogs and cats. For research applications, suitable subjects may include mammals, such as rodents (e.g., mice, rats, hamsters), rabbits, primates, and swine such as inbred pigs and the like.

The term “treatment” or “treating” refers to protection of a subject from a disease, such as preventing, suppressing, repressing, ameliorating, or completely eliminating the disease. Preventing the disease involves administering a conjugate of the present disclosure to a subject prior to onset of the disease. Suppressing the disease involves administering a conjugate of the present disclosure to a subject after induction of the disease but before its clinical appearance. Repressing or ameliorating the disease involves administering a conjugate of the present disclosure to a subject after clinical appearance of the disease.

### 2. Conjugates

Disclosed herein are conjugates that include a biologically active agent and a plurality of POEGMA molecules conjugated to the biologically active agent. It has been found that by conjugating a high density of POEGMA molecules to a biologically active agent, the overall pharmacokinetics of the conjugate can be improved and its immune response can be reduced or eliminated—compared to a PEG-biologically active agent conjugate. The reduced or eliminated immune response can include both a reduced or eliminated antigenicity and a reduced or eliminated immunogenicity of the disclosed biologically active agent-POEGMA conjugate. Accordingly, the disclosed conjugate can have beneficial interactions with a subject's immune system.

The beneficial immune interactions of the conjugate can also be seen in that the conjugate may not induce an anti-POEGMA antibody response. An anti-POEGMA antibody response can include inducing IgG class antibodies, inducing IgM class antibodies, inducing IgE class antibodies, inducing IgA class antibodies, or a combination thereof. Accordingly, in some embodiments, the conjugate does not induce anti-POEGMA IgG class antibodies, anti-POEGMA IgM class antibodies, anti-POEGMA IgE class antibodies, anti-POEGMA IgA class antibodies, or a combination thereof. In some embodiments, the conjugate does not induce anti-POEGMA IgG class antibodies and/or anti-POEGMA IgM class antibodies. In addition, the conjugate may not be reactive with anti-PEG antibodies. In some embodiments, the conjugate is not reactive with pre-existing anti-PEG antibodies in a subject. The immune properties of the disclosed conjugates can be assessed as described in the examples below.

With respect to the PEG-biologically active agent conjugate, this conjugate can be considered a control as to what the disclosed conjugate is compared to when assessing reducing or eliminating antigenicity, immunogenicity, or both. The control can be of similar molecular weight. The control can also be branched or linear, as long as it has more than the disclosed number of consecutive ethylene glycol monomers in tandem. For example, a suitable control PEG can include linear or branched PEG having more than 9 consecutive ethylene glycol monomers in tandem.

The control can also have a similar amount of PEG molecules (relative to POEGMA molecules) conjugated to the biologically active agent. For example, the disclosed conjugate can have a reduced immune response relative to a PEG-biologically active agent conjugate having about 5 to about 130 PEG molecules per biologically active agent, such as about 10 to about 120 PEG molecules per biologically active agent, about 15 to about 100 PEG molecules per biologically active agent, about 20 to about 80 PEG molecules per biologically active agent, about 10 to about 50 PEG molecules per biologically active agent, about 15 to about 40 PEG molecules per biologically active agent, about 10 to about 35 PEG molecules per biologically active agent, about 20 to about 30 PEG molecules per biologically active agent, or about 25 to about 30 PEG molecules per biologically active agent. In some embodiments, the disclosed conjugate has a reduced immune response relative to a PEG-biologically active agent conjugate having about 30 PEG molecules per biologically active agent. In some embodiments, the biologically active agent of the control is uricase.

In addition to the advantageous immune system properties, the disclosed conjugates can also have improved pharmacokinetics. For example, the conjugate can have a t_{1/2} elimination of greater than or equal to 45 h; a Cₒ of at least 45 nM, an AUC of at least 2700 nM×h, or a combination thereof. In addition, the disclosed conjugate can have improved pharmacokinetics compared to a PEG-biologically active agent control as described herein (e.g., a PEG-biologically active agent conjugate having about 5 to about 130 PEG molecules per biologically active agent). For example, the conjugate can have a Cₒ of at least 1.1 times greater than a Cₒ of a PEG-biologically active agent conjugate control; a t_{1/2} elimination of at least 1.3 times greater than a t_{1/2} elimination of a PEG-biologically active agent conjugate control; an AUC of at least 1.4 times greater than an AUC of a PEG-biologically active agent conjugate control; or a combination thereof. The pharmacokinetic profile of the disclosed conjugates can be assessed as described in the examples below.

The conjugate may have a varying hydrodynamic size (Rₕ) due, in part, to the biologically active agent and the POEGMA molecules. For example, the conjugate can have a hydrodynamic size of about 2 nm to about 12 nm, such as about 3 nm to about 10 nm or about 4 nm to about 9 nm. In some embodiments, the conjugate has a hydrodynamic size of greater than about 8.6 nm, which can be useful to avoid renal excretion. Hydrodynamic size can be measured by techniques used within the art, such as dynamic light scattering.

### A. Biologically Active Agents

The conjugate includes a biologically active agent. A large variety of different biologically active agents may be used with the high density POEGMA of the disclosure. Examples include, but are not limited to, a monoclonal antibody, blood factor, betatrophin, exendin, enzyme, asparaginase, glutamase, arginase, arginine deaminase, adenosine deaminase (ADA), ADA-2, ribonuclease, cytosine deaminase, trypsin, chymotrypsin, papain, growth factor, epidermal growth factor (EGF), insulin, insulin-like growth factor (IGF), transforming growth factor (TGF), nerve growth factor (NGF), platelet-derived growth factor (PDGF), bone morphogenic protein (BMP), fibroblast growth factor (FGF), somatostatin, somatotropin, somatropin, somatrem, calcitonin, parathyroid hormone, colony stimulating factors (CSF), clotting factors, tumor necrosis factors (TNF), gastrointestinal peptides, vasoactive intestinal peptide (VIP), cholecystokinin (CCK), gastrin, secretin, erythropoietins, growth hormone, GRF, vasopressins, octreotide, pancreatic enzymes, superoxide dismutase, thyrotropin releasing hormone (TRH), thyroid stimulating hormone, luteinizing hormone, luteinizing hormone-releasing hormone (LHRH), growth hormone releasing hormone (GHRH), tissue plasminogen activators, interleukins, interleukin-1, interleukin-15, interleukin-2, interleukin-10, colony stimulating factor, granulocyte macrophage colony-stimulating factor (GM-CSF), interleukin-1 receptor antagonist (IL-1RA), glucagon-like peptide-1 (GLP-1), exenatide, GLP-1 R multi-agonist, GLP-1 R antagonist, GLP-2, TNF-related apoptosis-inducing ligand (TRAIL), leptin, ghrelin, granulocyte monocyte colony stimulating factor (GM-CSF), interferons, interferon-α, interferon-gamma, human growth hormone (hGH) and antagonist, macrophage activator, chorionic gonadotropin, heparin, atrial natriuretic peptide, hemoglobin, relaxin, cyclosporine, oxytocin, vaccines, monoclonal antibodies, single chain antibodies, ankyrin repeat proteins, affibodies, activin receptor 2A extracellular domain, alpha-2 macroglobulin, alpha-melanocyte, apelin, bradykinin B2 receptor antagonist, cytotoxic T-lymphocyte-associated protein (CTLA-4), elafin, Factor IX, Factor VIIa, Factor VIII, hepcidin, infestin-4, kallikrein inhibitor, L4F peptide, lacritin, parathyroid hormone (PTH), peptide YY (PYY), thioredoxin, thymosin B4, uricase, urodilatin, aptamers, silencing RNA, microRNA, long non-coding RNA, ribozymes, analogs and derivatives thereof, and combinations thereof.

In some embodiments, the biologically active agent includes a nucleotide, a polynucleotide, a protein, a peptide, a polypeptide, a carbohydrate, a lipid, a small molecule drug, or a combination thereof. In some embodiments, the biologically active agent includes a nucleotide, a polynucleotide, a protein, a peptide, or a polypeptide. In some embodiments, the biologically active agent includes a protein, a peptide, or a polypeptide. In some embodiments, the biologically active agent includes a protein. In some embodiments, the biologically active agent includes uricase.

### i. Uricase

In some embodiments, the biologically active agent is uricase. Uricase is a tetrameric protein including four identical monomers that can catalyze the oxidation of uric acid to allantoin, hydrogen peroxide, and carbon dioxide. Uric acid has a complex physiological role in various processes, including inflammation and danger signaling. Further, modern purine-rich diets can lead to hyperuricemia, which is linked to many diseases including an increased risk of developing gout. Accordingly, uricase-and its ability to catalyze the oxidation of uric acid—can be used in methods of treating diseases that can be affected by uric acid, such as gout.

The uricase can have limited aggregation. For example, the uricase and/or conjugate may be essentially free of uricase aggregates. In some embodiments, the uricase and/or conjugate is free of uricase aggregates. In addition, the uricase and/or conjugate can have a limited amount of uricase octamer present. For example, the uricase and/or conjugate can have less than 1.5% octamer by mass of uricase, less than 1% octamer by mass of uricase, less than 0.9% octamer by mass of uricase, less than 0.8% octamer by mass of uricase, less than 0.7% octamer by mass of uricase, less than 0.6% octamer by mass of uricase, or less than 0.5% octamer by mass of uricase.

The uricase may maintain activity when conjugated to the POEGMA molecules. For example, the conjugate may have a uricase activity of about 10 U/mg uricase to about 20 U/mg uricase, such as about 10 U/mg uricase to about 18 U/mg uricase, about 10.5 U/mg uricase to about 16 U/mg uricase, or about 11 U/mg uricase to about 14 U/mg uricase.

### B. POEGMA

The POEGMA can instill the conjugate with advantageous stealth and immune system properties. The POEGMA has a poly(methyl methacrylate) backbone and a plurality of side chains covalently attached to the backbone. The side chains are oligomers of ethylene glycol (EG). For example, each side chain can include 2 to 9 monomers of EG repeated in tandem, such as 2 to 8 monomers of EG repeated in tandem, 2 to 7 monomers of EG repeated in tandem, 2 to 6 monomers of EG repeated in tandem, 2 to 5 monomers of EG repeated in tandem, or 2 to 4 monomers of EG repeated in tandem. In some embodiments, each side chain includes 3 monomers of EG repeated in tandem.

The conjugate can include the POEGMA at high densities without inducing an adverse immune response. The conjugate can include about 5 to about 130 POEGMA molecules per biologically active agent, such as about 10 to about 120 POEGMA molecules per biologically active agent, about 15 to about 100 POEGMA molecules per biologically active agent, about 20 to about 80 POEGMA molecules per biologically active agent, about 10 to about 50 POEGMA molecules per biologically active agent, about 15 to about 40 POEGMA molecules per biologically active agent, about 10 to about 35 POEGMA molecules per biologically active agent, about 20 to about 30 POEGMA molecules per biologically active agent, or about 25 to about 30 POEGMA molecules per biologically active agent.

In some embodiments, the conjugate includes greater than 5 POEGMA molecules per biologically active agent, greater than 6 POEGMA molecules per biologically active agent, greater than 7 POEGMA molecules per biologically active agent, greater than 8 POEGMA molecules per biologically active agent, greater than 9 POEGMA molecules per biologically active agent, greater than 10 POEGMA molecules per biologically active agent, greater than 15 POEGMA molecules per biologically active agent, greater than 20 POEGMA molecules per biologically active agent, or greater than 25 POEGMA molecules per biologically active agent.

In some embodiments, the conjugate includes less than 100 POEGMA molecules per biologically active agent, less than 90 POEGMA molecules per biologically active agent, less than 80 POEGMA molecules per biologically active agent, less than 70 POEGMA molecules per biologically active agent, less than 60 POEGMA molecules per biologically active agent, less than 50 POEGMA molecules per biologically active agent, less than 40 POEGMA molecules per biologically active agent, less than 35 POEGMA molecules per biologically active agent, or less than 30 POEGMA molecules per biologically active agent.

Adjacent side chains may be the same within the same POEGMA molecule or they may be different. For example, one side chain may have 3 monomers of EG repeated in tandem, while another side chain (in the same POEGMA molecule) may have 4 monomers of EG repeated in tandem.

Each side chain can have a first terminal end and a second terminal end. The first terminal end can be covalently attached to the backbone. The second terminal end can be free. The second terminal end may be modified. In some embodiments, each second terminal end independently includes an alkyl, ester, amine, amide, or carboxyl group. In some embodiments, each second terminal end includes an alkyl. In some embodiments, each second terminal end includes a C₁-C₄ alkyl. In some embodiments, each second terminal end includes a methyl group. In some embodiments, each second terminal end does not include a hydroxyl group.

The second terminal end of each side chain may be the same or different from the second terminal end of an adjacent side chain in the same POEGMA molecule. In some embodiments, the second terminal end of each side chain is the same throughout the POEGMA. In some embodiments, the second terminal end of at least one side chain is different from the second terminal end of at least one adjacent side chain.

In addition, the backbone can have a first terminal end and a second terminal end.

The POEGMA can have a varying molecular weight. For example, each POEGMA molecule can independently have a weight average molecular weight of about 1,000 Da to about 100,000 Da, such as about 2,000 Da to about 90,000 Da, about 3,000 Da to about 80,000 Da, about 4,000 Da to about 70,000 Da, about 5,000 Da to about 60,000 Da, about 6,000 Da to about 50,000 Da, about 7,000 Da to about 40,000 Da, about 8,000 Da to about 30,000 Da, or about 9,000 Da to about 20,000 Da. In some embodiments, each POEGMA molecule independently has a weight average molecular weight of about 10,000 Da. Molecular weight of the POEGMA can be measured by techniques used within the art, such as SEC, SEC combined with multi-angle light scattering, gel permeation chromatography, and the like.

Further discussion on POEGMA, its synthesis, and it application can be found in U.S. Pat. Nos. 8,497,356 and 10,364,451, both of which are incorporated herein by reference in their entirety.

### C. Synthesis of Conjugates

Also disclosed are methods of making the conjugates. The method can include conjugating the POEGMA molecules, each molecule having a poly(methyl methacrylate) backbone and a plurality of side chains covalently attached to the backbone, each side chain comprising 2 to 9 monomers of EG repeated in tandem to the biologically active agent to provide the conjugate.

Each POEGMA molecule can be conjugated to the biologically active agent through at least one of its side chains, its backbone, or a combination thereof. In some embodiments, the biologically active agent is conjugated to a side chain of each POEGMA molecule or the backbone of each POEGMA molecule. In some embodiments, the biologically active agent is conjugated to a side chain of a first set of POEGMA molecules and to a backbone of a second set of POEGMA molecules. In some embodiments, the biologically active agent is conjugated to the backbone of each POEGMA molecule. In some embodiments, the biologically active agent is conjugated to a terminal end of the backbone of each POEGMA molecule. The POEGMA molecules can be conjugated to the biologically active agent in a non-site-specific manner.

The POEGMA molecules can be conjugated to the biologically active agent through any suitable conjugation strategy known within the art. For example, the biologically active agent and each POEGMA molecule may each individually have functional groups that are complimentary to each other in that they can form a covalent bond between the functional groups under appropriate conditions. Representative complimentary functional groups that can form a covalent bond include, but are not limited to, an amine and an activated ester, an amine and an isocyanate, an amine and an isothiocyanate, an amine and a carbonate, thiols for formation of disulfides, an aldehyde and amine for enamine formation, and an azide for formation of an amide via a Staudinger ligation. Depending on the functional groups, different bonds or linkages can be formed between the biologically active agent and the POEGMA molecules. In some embodiments, the biologically active agent is conjugated to each POEGMA molecule individually through a urethane bond.

Each POEGMA molecule can be functionalized at its backbone or at a side chain. In some embodiments, each POEGMA molecule is functionalized at a terminal end of its backbone. In some embodiments, each POEGMA molecule is functionalized with a hydroxyl group, carboxyl group, carbonate group, amine group, ester group, azide group, alkyne group, or a combination thereof. In some embodiments, each POEGMA molecule is functionalized with a carbonate group, a hydroxyl group, a carboxyl group, or an ester group. In some embodiments, each POEGMA molecule is functionalized with a carbonate group or a hydroxyl group. In some embodiments, each POEGMA molecule is functionalized with a carbonate group. In some embodiments, each POEGMA is functionalized with a nitrophenyl carbonate group.

In some embodiments, the biologically active agent is uricase and uricase is not functionalized. For example, uricase has 128 lysines present in a tetramer. Accordingly, uricase can have up to 128 conjugation sites for a POEGMA molecule even when not functionalized. In some embodiments, uricase is conjugated to each POEGMA via a lysine through, e.g., a urethane bond. In some embodiments, each POEGMA is functionalized at a terminal end of its backbone with a nitrophenyl carbonate group and conjugated to a lysine of uricase through a urethane bond.

The uricase may be modified prior to conjugation in a manner that minimizes aggregate formation. Recombinant uricase from commercial suppliers can include up to about 86% aggregates, which can prohibit its utility in the conjugate synthesis. The uricase disclosed herein can overcome this issue by being expressed as part of a fusion protein with an elastin-like polypeptide (ELP). For example, an ELP can be fused to the C-terminus of uricase monomers at the gene level. A protease cleavage site can be inserted in between the ELP and the uricase monomer, such as tobacco etch virus (TEV). The protease cleavage site can allow the liberation of the uricase monomer from the ELP after expression. In some embodiments, uricase is expressed recombinantly in bacterial expression systems, such as E. coli.

The method can further include purifying the conjugate by techniques known within the art, such as SEC. In addition, the ELP can be used to perform inverse transition cycling (ITC) in purification methods of the fusion protein.

The description of the conjugates, biologically active agent, uricase, and POEGMA can also be applied to the methods of making the conjugate.

### 3. Methods

### A. Methods of Reducing Immunogenicity

Also disclosed are methods of reducing immunogenicity of a polymer-biologically active agent conjugate. For example, the disclosed methods can reduce the immunogenicity of a PEG-biologically active agent conjugate by replacing PEG with the disclosed POEGMA molecules.

The method can include conjugating a plurality of POEGMA molecules to the biologically active agent to form a biologically active agent-POEGMA conjugate as disclosed herein. For example, the method can include conjugating about 5 to about 130 POEGMA molecules to the biologically active agent, each POEGMA molecule having a poly(methyl methacrylate) backbone and a plurality of side chains covalently attached to the backbone, each side chain comprising 2 to 9 monomers of EG repeated in tandem to provide a conjugate, wherein the conjugate has a reduced immune response relative to a PEG-biologically active agent conjugate having about 5 to about 130 PEG molecules per biologically active agent.

Because the methods of reducing immunogenicity include conjugating the biologically active agent to a plurality of POEGMA molecules, the description of the methods of making the disclosed conjugates can also be applied to the methods of reducing immunogenicity. In addition, the description of the conjugates, biologically active agent, uricase, and POEGMA can also be applied to the methods of reducing immunogenicity.

### B. Methods of Treating Diseases

The present disclosure also provides methods of treating a disease. The methods can include administering to a subject (in need thereof) an effective amount of the conjugate as detailed herein. In some embodiments, the disease is affected by uric acid. In some embodiments, the disease includes gout. Further discussion on the administration and formulation of POEGMA-based conjugates can be found in PCT/US2022/023158, which is incorporated herein by reference in its entirety.

The disclosed invention has multiple aspects, illustrated by the following non-limiting examples.

### 4. EXAMPLES

### Example 1

### Materials & Methods

Gene cloning. The amino acid sequence for Candida utilis uricase (Acc. No.: A0A1E4S4E2) was codon-optimized for Escherichia coli (E. coli), synthesized (Integrated DNA Technologies, Coralville, IA), and cloned into a modified PET-24a+ vector using restriction digest with AcuI and BseRI. A previously described seamless cloning method was used to clone in a C-terminal TEV protease recognition site (ENLYFQ; t), followed by an ELP, including 60 repeats of VPGVP pentamer. The final construct encoding UTE was transformed into and expressed by BL21-DE3 E. coli. Next, an ELP-tagged TEV protease was cloned. Briefly, the gene encoding for TEV protease (Acc. No.: QOGDU8, residues 73-302) was PCR-amplified and cloned into PET-24a+ via restriction digest. A six-residue N-terminal His-tag (His₆) and an ELP including 60 repeats of VPGVG pentamer were cloned onto the TEV protease gene to generate the His₆-ELP-TEV construct. The resulting vector was transformed into BL21-DE3 E. Coli.

Protein expression and purification. UTE-expressing E. coli cultures were grown in shake flasks at 25° C. for 4 hours in 2XYT media, induced with 0.5 mM isopropyl β-D-1-thiogalactopyranoside (IPTG), and grown overnight at 16° C. Cells were harvested through centrifugation at 3400 g and 4° C. for 10 minutes and resuspended in cold borate buffer (100 mM sodium borate 1 mM ethylenediaminetetraacetic acid EDTA pH 10). The cells were lysed via sonication (Q500 sonicator, QSonica, Newtown, CT) pulsed at 10 s on and 30 s off for 180 s of total sonication time. DNA precipitates were generated by adding 10% polyethyleneimine (PEI), and lysates were clarified by centrifugation at 24000 g and 4° C. for 10 minutes. UTE was purified from the clarified lysate by ITC. Briefly, the lysate was brought to room temperature, and the aggregation of UTE was triggered by the addition of 0.1 M ammonium sulfate. The aggregates were pelleted by centrifugation at 24,000 g and 35° C. for 10 minutes. The pellets were resuspended in cold borate buffer on a rotator at 4° C. for 1 hour, and contaminants were removed via centrifugation at 24,000 g and 4° C. for 10 minutes. This process was repeated for one or two cycles to reach UTE with purity greater than 95%. His₆-ELP-TEV was expressed, harvested, lysed, and clarified as described above for UTE, followed by purification by nickel affinity chromatography and dialysis into Tris-EDTA buffer. Purity was determined by SDS-PAGE using gel densitometry analysis (Biorad). Protein concentration was determined by UV-vis spectroscopy using an ND-1000 Nanodrop spectrometer (Thermo Scientific).

TEV protease cleavage. UTE was cleaved by TEV protease to remove ELP and produce free uricase. Briefly, the reaction included a 1:10 ratio of His₆-TEV-ELP to UTE by absorbance at 280 nm in borate reaction buffer (100 mM sodium borate pH 9.2, 1 mM EDTA, and 10 mM dithiothreitol). The reaction was incubated on a rotator for 20 hours at 4° C. Finally, the His₆-ELP-TEV, uncleaved UTE, and cleaved ELP were all purified out by adding 0.2 M ammonium sulfate to trigger ELP aggregation, followed by centrifugation at 24,000 g and 25° C. for 10 minutes. The resulting free tetrameric uricase was solubilized in borate buffer and dialyzed into a 50 mM carbonate buffer containing 100 mM NaCl pH 9.2 using a dialysis cassette with a molecular weight cut-off (MWCO) of 10 kDa. The purity was determined by SDS-PAGE using gel densitometry analysis (Biorad).

Synthesis of a polymerization initiator. 4-amino-1-butanol (5 g, 0.0561 mol) and triethylamine (TEA) (6.24 g, 0.0624 mol) were transferred into a reaction flask (flask 1) and solubilized in 20 ml dichloromethane (DCM). α-bromoisobutyryl bromide (12.8 g, 0.0567 mol) was solubilized in 1 ml DCM in another flask (flask 2). The solution in flask 2 was drop-wise transferred into flask 1 by using a syringe under an inert atmosphere. The reaction was stirred for 16 h at room temperature. The resulting mixture was filtered, and the organic phase was collected. Next, 20 ml potassium hydroxide (5%) was added and stirred for 2 h. The organic phase was separated using a separatory funnel and washed with 1N NaOH, 1N HCl, and saturated NaCl, respectively. The organic phase was dried over anhydrous MgSO₄ and filtered to collect the organic phase, followed by evaporation of DCM under vacuum.

POEGMA synthesis and purification. POEGMA was synthesized using ARGET-ATRP. Triethylene glycol methyl ether methacrylate (EG3) was passed through an alumina column to remove the inhibitor. The inhibitor-free EG3 (10 mmol), the polymerization initiator (0.04 mmol), methanol (5.8 ml), and 100 mM NaCl (11.6 ml) were mixed in a flask (polymerization flask). A catalytic complex was prepared by mixing 8 molar equivalents of tris(2-pyridylmethyl) amine (TPMA) with the molar equivalent of copper II bromide (CuBr₂) in water. The catalytic complex (0.08 mmol TPMA; 0.01 CuBr₂) was transferred into the polymerization flask. The final volume was adjusted to 20 ml. In a separate flask, 64 mM ascorbic acid was prepared. Both flasks were purged with argon to remove oxygen. The polymerization was started with the addition of ascorbic acid into the polymerization flask at a rate of 1 μl per minute. The reaction was continued for 90 minutes. The resulting mixture was purified by passing it through an alumina column, followed by diethyl ether precipitation and evaporation under vacuum, yielding pure OH-POEGMA.

POEGMA chain-end NPC activation. OH-POEGMA (0.11 mmol) was dissolved in 5 ml anhydrous acetonitrile in a reaction flask (flask 1). In another flask (flask 2), bis(4-nitrophenyl) carbonate (2.15 mmol) was solubilized in 15 ml anhydrous acetonitrile and transferred into the flask 1. Finally, pyridine (5.16 mmol) was transferred into flask 1, and the final volume was adjusted to 21.5 ml using anhydrous acetonitrile. The resulting mixture was reacted for 16 hours at room temperature. The precipitate was removed using glass fiber GF/B Filters (Whatman), followed by evaporating acetonitrile under vacuum until 0.5 ml remained. The resulting polymer mixture was purified via diethyl ether purification and kept under a vacuum to remove solvents. The NPC-POEGMA was stored at −20° C. under an inert atmosphere.

NPC activation degree calculation. NPC activation degree was calculated using Equation 1, where the proportion of the hydrogens present in the benzene ring of NPC (Peak a (2 H) at 8.26 ppm; Peak B (2 H) at 7.38 pm) to the methylene protons (2 H; 4.0-4.4 ppm) resulted in the NPC end-activation degree per EG3 monomeric unit. The activation degree per POEGMA chain was found by multiplying the NPC end-activation degree per EG3 monomeric unit with the degrees of polymerization (DP) of POEGMA. The DP is the number of EG3 chains present in a POEGMA chain and calculated by dividing POEGMA M_{w} by M_{w} of EG3 monomer.

$$\% N P C activation = \frac{( {\int }_{8.25 ppm}^{8.29 ppm} peak a + {\int }_{7.35 ppm}^{7.39 ppm} peak b ) / 4}{( {\int }_{3.8 ppm}^{4.2 ppm} peak c ) / 2} * D P * 100 \qquad Equation 1$$

Structural characterization of POEGMA. The chemical structure of the polymerization initiator and POEGMAs was analyzed by nuclear magnetic resonance (NMR) spectroscopy. The compounds were dissolved in deuterated chloroform, and data were recorded using a 400 MHz Varian INOVA spectrometer. The data was analyzed using ACD/NMR software (ACD Labs). The NPC activation degree was defined as the % NPC present on the POEGMA chains and calculated.

Conjugate synthesis and purification. NPC-POEGMA or NPC-PEG was weighed in flasks, followed by adding uricase into the flasks at a final concentration of 5 mg ml⁻¹. The flasks were placed onto a rotator for 1 hour at room temperature, allowing uricase to react with NPC-functional polymers. 100-fold molar equivalent POEGMA was used in the synthesis of uricase-POEGMA. The uricase-PEG_{Rh} and uricase-PEG_{Mw} required 10 and 100 molar equivalents of NPC-PEG (Creative PEGworks, NC). The resulting conjugates were purified by a single round of SEC. Briefly, an AKTA Purifier equipped with a UV-Vis detector operating at 220 and 280 nm and a HiLoad 26/600 Superdex 200 pg column (GE Healthcare). The mobile phase was 10 mM carbonate buffer containing 100 mM NaCl. The flow rate was 2 ml min⁻¹. The purified conjugates were buffer exchanged into 10 mM borate buffer using desalting columns with a 40 kDa MWCO (Pierce), followed by storage at −80° C.

Physical characterization of the conjugates. Uricase variants were characterized for Mₙ, M_{w}, and Ð by SEC-MALS using an Agilent 1260 HPLC equipped with a DAWN HELEOS II MALS detector (Wyatt Technology), an Optilab T-rEX refractive index detector (Wyatt Technology), and a UV-vis detector operating at 280 nm (Agilent). The MALS detector was normalized using bovine serum albumin (BSA) before each use. The mobile phase was 10 mM carbonate buffer with 150 mM NaCl and 100 ppm NaN₃ (pH 10.3). The uricase variants were solubilized in the mobile phase, followed by filtration using 100 nm syringe filters (Whatman). 50 μl of the resulting solutions were separated on a WTC-015N5 SEC column (5 μm; 150 Å; 4.6 mm internal diameter; Wyatt Technology). The flow rate was 0.5 ml min⁻¹. The light scattering data were analyzed for Mₙ, M_{w}, Ð, and conjugation stoichiometry using ASTRA software (Wyatt Technology). The refractive index increment (dn/dc) and UV extinction coefficient were determined by the refractive index and UV-vis detectors, respectively. OH- and NPC-POEGMA was characterized for Mₙ, M_{w}, and Ð.

Hydrodynamic size characterization. The Rₕ was characterized by DLS using a DynaPro Plate Reader (Wyatt Technology). The uricase variants were buffer exchanged into PBS at pH 7.4 using Zeba desalting columns with 40 kDa MWCO (Pierce), followed by filtration through a 100 nm syringe filter (Whatman). The measurements were recorded at 15° C. Data were analyzed by applying a regularization fit for Raleigh spheres using Dynamics software (Wyatt Technology). In the stability experiment, uricase was cleaved from the ELP and buffer exchanged to PBS at pH 7.4 using a Zeba desalting column (Pierce), followed by collecting the DLS data at the specified time points.

Biochemical activity assay. The uricase variants' activity was quantified using Amplex™ Red Uric Acid/Uricase Assay Kit (Thermo Scientific). Briefly, uricase catalyzes the conversion of uric acid to allantoin, hydrogen peroxide, and carbon dioxide. In the presence of horseradish peroxidase (HRP), hydrogen peroxide reacts stoichiometrically with the Amplex Red reagent to generate a red dye, which can be monitored kinetically. Serial dilutions of the uricase variants were prepared and tested. Following the manufacturer's protocols, the uric acid conversion activity (U) per mg uricase present in the variants was reported.

Endotoxin purification. The conjugates were endotoxin purified using high-capacity endotoxin removal columns (Pierce) according to the manufacturer's protocols with minor changes. The mobile phase was 10 mM ammonium bicarbonate pH 7.5 dissolved in endotoxin-free water (Hyclone). The endotoxin-purified conjugates were lyophilized and stored at −80° C. The endotoxin content was tested using the Endosafe nexgen-PTS instrument and cartridges (Charles River). The maximum acceptable endotoxin limit was 0.2 EU per kg mouse body weight. The samples were sterilized before administration into mice using a 0.22 μm Acrodisc syringe filter (Pall Corporation).

Animal experiments. Six-week-old male C57BL/6J (Jackson Laboratories; stock no. 000664) were used for the in vivo studies. Mice were allowed to acclimate to the facilities for one week before the start of the experiments. Mice were group-housed in a photo-controlled environment with 12 h dark/light cycles and kept on a standard rodent diet with ad libitum access to water and food.

PK. The sterile and endotoxin-free uricase variants were labeled with a fluorophore to track the drug in vivo. The lack of free dye was confirmed by HPLC equipped with a fluorescence detector. The final concentration was set to 4.4 μM with endotoxin-free PBS.

The uricase variants were i.v. administrated into C57BL/6J mice (n=4-5 mice per group) at a dose of 36.6 nmol kg⁻¹. Blood samples (10 μl) were collected from a small nick on the tail vein to track plasma concentrations of the treatments into heparin-containing tubes (90 μl; 1000 U ml⁻¹). Time points for the blood sampling were pre-dose (−15-min), 40 sec, 5-min, 30-min, 1-, 2-, 4-, 8-, 24-, and every 24 hours up to 144 h. The samples were processed for plasma by centrifugation at 1,600 g for 15 minutes. The fluorophore concentration in the plasma samples was measured using a Victor plate reader (Perkin Elmer) at 485 nm (excitation) and 535 nm (emission) (n=3 wells per mouse). The plasma concentrations of the drugs were plotted as a function of sampling time, followed by fitting a one-phase decay curve using GraphPad Prism 9 software. The PK parameters were identified using a non-compartmental PK analysis. The drug concentration was approximated to the concentration at t=0 (Cₒ)—termed initial drug concentration—and calculated from the intersection of the fitted decay curve with the Y-axis at t=0. The elimination rate (k) was calculated from the slope of a linear regression curve fitted to the logarithm of drug concentration as a function of sampling time. The elimination half-life was defined as the time needed for the drug concentration to reach its half-maximal value and calculated using k.

The effect of the anti-drug antibodies on the drug PK was tested in a repeated PK assay as described above. The mice were weekly administrated with the same drugs a total of 5 times. Drug PK was tracked after the first injection and the fifth injection.

Immunogenicity. Sterile and endotoxin-free uricase-POEGMA and uricase-PEG_{Mw} were s.c. administered into separate cohorts of C57BL/6J mice (n=10) every 17 days three times at an equivalent dose of 36.6 nmol kg⁻¹ using equivalent injection volume of endotoxin-free PBS as a negative control. Next, ˜180 μl of blood were collected from the submandibular vein ten days after each injection into lyophilized heparin-containing tubes and processed to separate plasma as described above. The plasma samples were stored at −80° C. until ADA analysis.

Analysis of ADAs. ADAs were analyzed using a Luminex multiplexed assay. Briefly, the plasma samples were diluted 500-fold in PBS. Diluted plasma samples (50 μl) were transferred to a black 96-well-plate (Corning). Next, OVA-, OVA-PEG-, and OVA-POEGMA-coupled magnetic beads were added to each well (50 μl; 2500 beads per set), followed by 1 hour incubation on a plate shaker. The magnetic beads were separated on a magnetic ring, and wells were washed with 0.2% (w/v) I-Block (Thermo Scientific) in PBS (Hyclone)—termed assay buffer. To analyze IgG-class ADAs, R-Phycoerythrin-conjugated goat anti-mouse IgG (Jackson Immunoresearch; #115-115-164) was transferred to the wells (5 μg ml⁻¹; 100 μl) and incubated for 1 hour. To analyze IgM-class ADAs, biotin-conjugated goat anti-mouse IgM (Jackson Immunoresearch; #115-065-075) was used at the same concentration and volume and for the same duration. Next, the wells were washed with the assay buffer. For the analysis of IgM-class ADAs, streptavidin-R-phycoerythrin (SAPE; Invitrogen) was transferred into the wells (7.5 μg ml⁻¹; 100 μl) and incubated for 30 minutes, followed by washing the wells with the assay buffer. The beads were resuspended in 100 μl assay buffer, and their signal was measured by MAGPIX (Luminex).

Indirect and competitive ELISA. We probed the reactivity of uricase, uricase-PEG_{Mw}, and uricase-POEGMA towards PEG antibodies present in a murine plasma sample. A PEG antibody-positive murine plasma sample was available, where C57BL/6J mice (n=10) were repeatedly exposed to OVA-PEG emulsified in Freund's adjuvant, followed by blood collection and processing into plasma. The plasma samples collected from each mouse at the end of the study (Day 44) were mixed at equal volumes, resulting in a PEG-antibody positive plasma pool.

In indirect ELISA, first, 100 μl of OVA-PEG, uricase-PEG_{Mw}, and uricase-POEGMA solutions in carbonate buffer pH 9.2 were coated on 96-well-plate surface, such that there was 5 μg of PEG or POEGMA in each well. The uricase-coated wells matched with the conjugate-coated wells in the amount of uricase in each well. The antigen solutions were incubated overnight at 4° C. The wells were blocked with 1% (w/v) iBlock (Thermo Scientific) in PBS (Hyclone)—termed blocking buffer—for an hour, followed by PBS washes twice. Next, the pooled PEG antibody-positive murine plasma sample was diluted 500-fold in PBS, and 100 μl of the diluted plasma sample was transferred to each well, followed by incubation for an hour and PBS washes twice. A biotinylated goat anti-mouse IgM antibody (Jackson Immunoresearch; 115-065-075) was diluted in PBS (50 ng ml⁻¹), and 100 μl of the resulting solution was transferred to each well. The antibody solution was incubated for an hour and removed from the wells, followed by PBS washes twice. Streptavidin-poly HRP (Pierce) was diluted in PBS to 0.1 μg ml⁻¹ and transferred to the wells, followed by a 30 minute incubation. The excess solution was removed, and the wells were washed with PBS. TMB substrate (Pierce) was added to wells (50μl) and incubated for 10 minutes, followed by being stopped with the addition of 2N sulfuric acid (50 μl) and absorbance reading on a plate reader (Eppendorf) at 450 nm. The absorbance was measured using a Victor plate reader (Perkin Elmer).

In competitive ELISA, uricase, uricase-PEG_{Mw}, and uricase-POEGMA competed with an unrelated PEGylated drug (exendin-PEG) for binding to the pooled PEG-antibody positive murine plasma sample. Exendin-PEG was a stoichiometric conjugate of PEG with an M_{w} of 10.2 kDa. Exendin-PEG was coated on the surface of 96-well-plates to yield 5 μg of PEG per well by overnight incubation at 4° C. The murine plasma sample was diluted 250-fold in PBS. Uricase, uricase-PEG_{Mw}, and uricase-POEGMA were prepared at varying concentrations up to 40 μM. 250 μl of the resulting solutions were mixed with 250 μl of the diluted plasma, followed by overnight incubation on a rotator at 4° C. On the day of the assay, exendin-PEG was removed from the wells, and the wells were blocked with the blocking buffer for 1 hour at room temperature, followed by PBS washes twice. 100 μl of the resulting antigen: plasma mixtures were transferred to each well and incubated for an hour. The drugs were removed from the wells, followed by PBS washes twice. Next, a biotinylated goat anti-mouse IgM antibody (Jackson Immunoresearch; 115-065-075) was diluted in PBS (50 ng ml⁻¹), and 100 μl of the resulting solution was transferred to each well. The antibody solution was incubated for an hour and removed from the wells, followed by PBS washes twice. Streptavidin-poly HRP (Pierce) was diluted in PBS to 0.1 μg ml⁻¹ and transferred to the wells, followed by a 30 minute incubation. The excess solution was removed, and the wells were washed with PBS. TMB substrate (Pierce) was added to wells (50 μl) and incubated for 10 minutes, followed by being stopped with the addition of 2 N sulfuric acid (50 μl) and absorbance reading on a plate reader (Eppendorf) at 450 nm. The absorbance was measured using a Victor plate reader (Perkin Elmer).

Statistical analyses. Mice were randomly distributed into groups. The sample size was estimated using open-source G-power software. The experiments that do not involve animals were replicated at least twice and repeated at least three times. The PK experiments necessitated at least n=3 mice per group, while n=10 mice per treatment were used in the immunogenicity experiments. The data represented the mean response in the treatment group±standard error of the mean (SEM) unless otherwise noted. The data were analyzed using two-way ANOVA, followed by post-hoc Tukey's multiple comparison tests. p<0.05 indicated a statistically significant test. (*p<0.05; **p<0.01; ***p<0.001; ****p<0.0001). Not significant (ns). GraphPad Prism 9.0 was used for all statistical analyses.

### Example 2

Aggregate-Free Uricase Expression with High Yield and Pharmacological Activity

Uricase is a tetrameric protein comprised of four identical monomers. Uricase is highly aggregation-prone because of its high content (˜33%) and non-uniform distribution of hydrophobic amino acids on the surface, leading to strong intermolecular hydrophobic interactions and aggregation. The uricase aggregates in the final drug formulation are a significant problem because they reduce the pharmacological activity and induce a robust anti-drug antibody (ADA) response, accelerating the drug clearance and rendering the treatment ineffective only after a few injections. Recombinant uricase from commercial suppliers comprises up to 86% aggregates, prohibiting its utility in the conjugate synthesis.

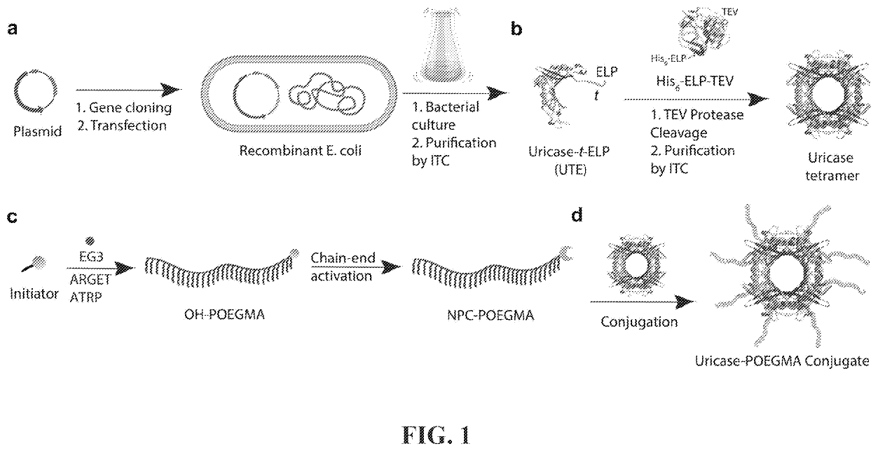

This problem was circumvented by exploiting stimuli-responsive elastin-like polypeptides (ELP) as a fusion protein to stabilize uricase during recombinant expression in E. coli. We fused an ELP to the C-terminus of the uricase monomers at the gene level (FIG. 1A). A Tobacco Etch Virus (TEV) protease recognition sequence—ENLYFQS was also inserted; termed t—between the ELP and uricase monomer so that TEV protease cleavage liberates the uricase monomers from the ELP after bacterial expression. The resulting fusion protein—termed uricase-t-ELP (UTE)—was recombinantly expressed in E.coli with ˜200 mg per liter of shaker flask culture yield. Furthermore, we exploited ELP's stimuli-responsive phase behavior for chromatography-free purification of UTE from the bacterial cell lysate by inverse transition cycling (ITC), yielding pure UTE, as assessed by sodium dodecyl sulfate-polyacrylamide gel electrophoresis (SDS-PAGE).

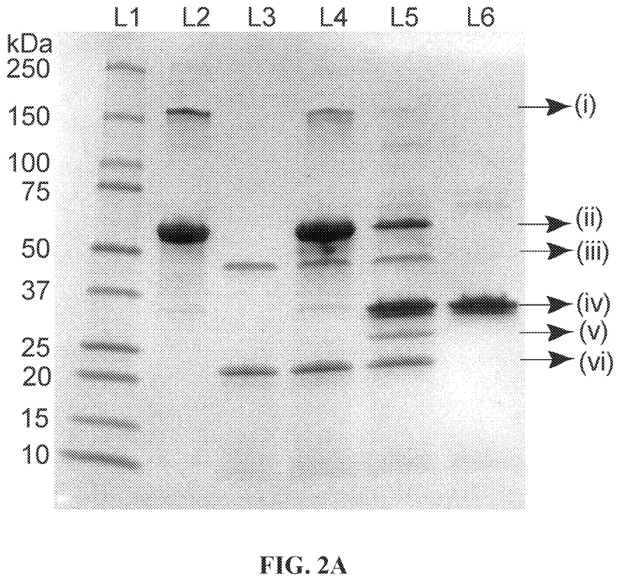

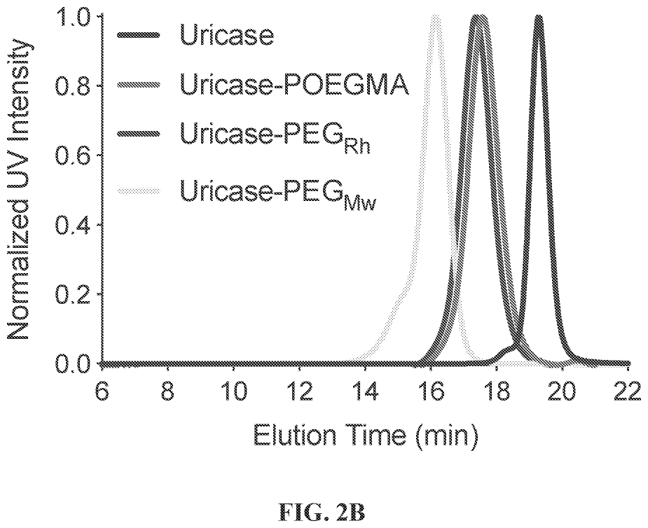

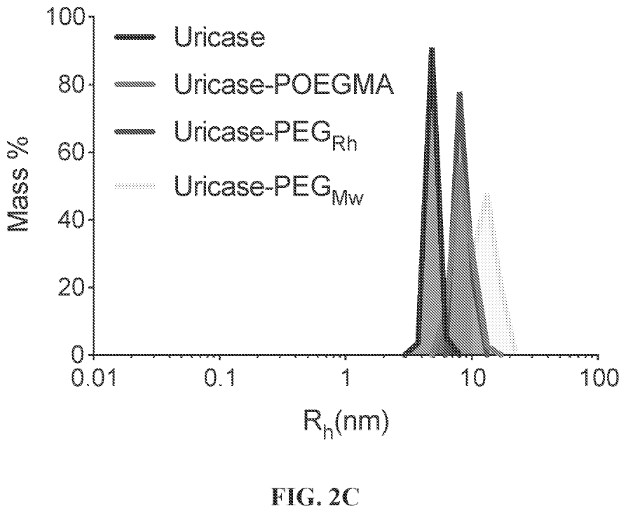

TEV protease was also recombinantly expressed in E.coli as a C-terminus ELP fusion with an N-terminus histidine (His) tag—termed His₆-ELP-TEV. It cleaved UTE between the Q and S amino acids and liberated uricase-ENLYFQ from the ELP with ˜72% yield, allowing uricase tetramers to form (FIG. 1B and FIG. 2A). Tetrameric uricase was then purified from His₆-ELP-TEV, unreacted UTE, and cleaved ELP by a single round of ITC as it was the only moiety that did not carry an ELP, yielding pure uricase tetramers (FIG. 2A).

Uricase tetramer had a number-averaged molecular weight (Mₙ) of 136.6 kDa, as measured by size-exclusion chromatography-multi-angle light scattering (SEC-MALS) (FIG. 2B; Table 1). Uricase had a hydrodynamic size (Rₕ) of ˜4.7 nm (FIG. 2C; Table 1), as measured by dynamic light scattering (DLS). Uricase comprised of 0.8 mass % octamer, a less-frequent native conformation. It was free of aggregates, indicated by the lack of larger molecules, confirmed by SEC-MALS and DLS.

Uricase's pharmacological activity was quantified using a biochemical assay, where it catalyzed the conversion of its natural substrate—uric acid—to highly soluble allantoin while producing hydrogen peroxide and carbon dioxide as byproducts. Our expression and purification protocol yielded uricase with an activity of ˜12 U/mg (Table 1), significantly higher than uricase from a commercial supplier (Sigma) with an activity of 4.4 U/mg. In addition, uricase was stable for up to one week at 4° C. in phosphate-buffered saline (PBS), indicated by >98 mass % of the molecules retained their original size and activity. Overall, we developed a protocol to express aggregate-free uricase with high yield and stability and purify without labor-intensive chromatography methods.

### Example 3

### Synthesis and Characterization of Uricase-POEGMA Conjugates

We synthesized POEGMA using a hydroxyl-functional polymerization initiator and three ethylene glycol-long (EG3) monomers using activators regenerated by electron transfer atom transfer radical polymerization (ARGET-ATRP), yielding hydroxyl-functional POEGMAs (OH-POEGMA). The OEG side-chain length of the resulting OH-POEGMA was confirmed by nuclear magnetic resonance (NMR) spectroscopy. The resulting OH-POEGMA was monodisperse with a low polydispersity (Ð) of <1.2 and had a weight-averaged molecular weight (M_{w}) of ˜10 kDa, as analyzed by gel permeation chromatography-MALS (GPC-MALS). This M_{w} was chosen to match pegloticase's structure, where 10 kDa PEG was used for conjugation.

We synthesized POEGMA conjugates of uricase by coupling nitrophenyl carbonate (NPC) functional POEGMA (NPC-POEGMA) to the amine residues on the exposed lysine amino acids in a non-site-specific and non-stoichiometric manner, resulting in a physiologically stable urethane bond. We synthesized NPC-POEGMA by activating the hydroxyl end group of OH-POEGMA to NPC. The NPC chain activation was confirmed by NMR spectroscopy and did not alter POEGMA's Mₙ, M_{w}, and Ð.

The resulting conjugate had an average of ˜27 POEGMA chains per uricase tetramer (FIG. 2B; Table ₁). The conjugation stoichiometry was chosen to approximate the pegloticase structure, comprising ₃₂ PEG chains on average per uricase tetramer that shield uricase's immunogenic epitopes distributed over its surface and improve the drug PK. We also synthesized a uricase-PEG conjugate with matching conjugation stoichiometry and M_{w}—termed uricase-PEG_{Mw}—to use as a control. The conjugates were aggregate-free and monodisperse with a low Ð of <1.2 and had near-identical conjugation stoichiometries (FIG. 2B; Table 1). Despite this, the conjugates significantly differed in their Rh (FIG. 2C; Table 1) due to POEGMA's more compact brush architecture than amorphous linear PEG. Given that the renal extraction rate may be affected by the Rₕ of the conjugates and complicate head-to-head PK comparisons, we also synthesized an Rₕ-matching uricase-PEG conjugate-termed uricase-PEG_{Rh}. The uricase-PEG_{Rh} was monodisperse and had a conjugation stoichiometry of ˜9 (FIG. 2C; Table 1).

The conjugates did not differ in enzymatic activity and were as effective as uricase, indicating that PEG or POEGMA conjugation did not affect the pharmacological activity (Table 1). Conversely, PEG and POEGMA coupling resulted in several degrees of magnitude decrease in activity when a large biological moiety, such as membrane-bound receptors, was targeted. We attributed this result to the small size of uric acid as a substrate, which should penetrate through PEG and POEGMA chains to reach uricase. POEGMA-attached asparaginase conjugates also showed only a minimal (3-fold) change in activity to catalyze its small molecule substrate asparagine, supporting our findings.

Table 1: Summary of conjugate characterization. The Mₙ, M_{w}, and Ð were measured by SEC-MALS given in FIG. 2B. Conjugation stoichiometry was calculated by SEC-MALS (n=3). The Rₕ was measured by DLS given in FIG. 2C (n=10). The activity values were derived from a biochemical assay (n=5) measuring the ability of the uricase to convert uric acid into allantoin. Data were reported as mean±standard deviation. Not applicable (N/A).

| | | | | Conjugation | | |
|---|---|---|---|---|---|---|
| | Mₙ | M_{w} | | Stoichiometry | | Activity |
| Compound | (kDa) | (kDa) | Ð | (Polymer: Uricase) | Rₕ (nm) | (U/mg uricase) |
| Uricase | 136.6 | 136.6 | 1.00 | N/A | 4.7 ± .02 | 11.6 ± 1.3 |
| Uricase-POEGMA | 417.5 | 426.1 | 1.09 | 27.0 ± 1.4 | 8.6 ± 1.5 | 12.1 ± 0.5 |
| Uricase-PEG_{Rh} | 228.3 | 252.7 | 1.10 | 8.9 ± 1.1 | 8.9 ± 0.3 | 10.8 ± 0.2 |
| Uricase-PEG_{Mw} | 440.6 | 443.6 | 1.11 | 29.3 ± 1.7 | 14.6 ± 1.5 | 11.4 ± 0.2 |

### Example 4

### Pharmacokinetics (PK)

We next investigated uricase-POEGMA's PK and compared it with unmodified uricase, uricase-PEG_{Mw}, and uricase-PEG_{Rh}. The uricase variants were fluorescently labeled to track them in vivo. We confirmed that uricase-POEGMA would not phase transition upon administration. The phase transition temperature (Tₜ) was above body temperature (˜42° C.) at the injection concentration in PBS. The uricase variants were intravenously (i.v.) administrated into 6-week-old male C57BL/6J mice (n=5), followed by tracking plasma drug concentration and calculating PK parameters.

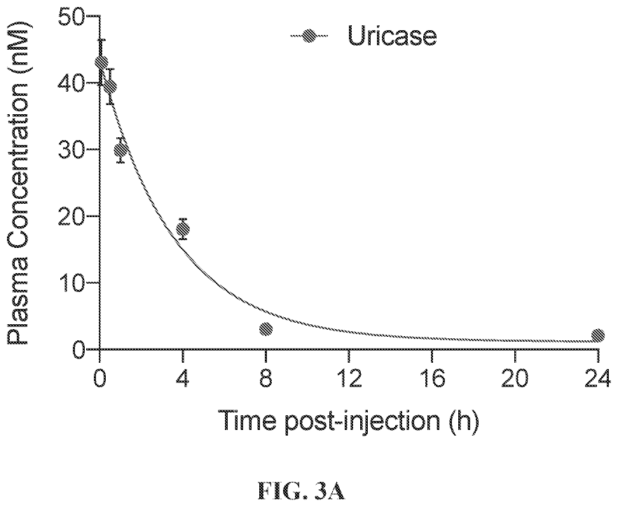

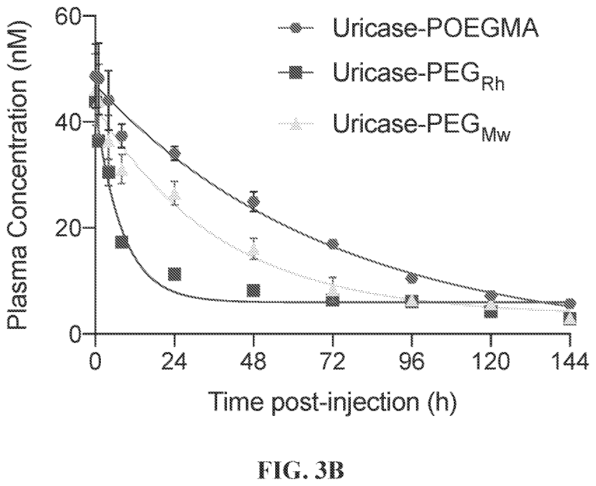

The treatments resulted in near-constant initial plasma concentration (Cₒ), indicating successful equimolar drug administration (Table 2). Unmodified uricase had a short elimination half-life (t_{1/2 elimination}) (FIG. 3A; Table 2). All conjugates had longer t_{1/2 elimination} than unmodified uricase, indicating that both PEG and POEGMA successfully extended uricase's PK (FIG. 3B; Table 2). Uricase-PEG_{Rh} showed bi-phasic PK, indicated by faster clearance upon administration, followed by a slower clearance of the remaining drug. We hypothesize, without being bound by a particular theory, that this could be due to its precipitation in circulation, given that it has the lowest conjugation stoichiometry among the conjugates, which could potentially negatively impact its stability. The PEG conjugates significantly differed in t_{1/2 elimination}. Uricase-PEG_{Mw} prolonged uricase's circulation by ˜16-fold (t_{1/2 elimination} of 35.5 h vs. 2.2 h), while uricase-PEG_{Rh} had only a ˜6-fold longer circulation than uricase (t_{1/2 elimination} of 13.1 h vs. 2.2 h). Uricase-PEG_{Rh} had much lower exposure than uricase-PEG_{Mw}, indicated by the lower area under the curve (AUC) (Table 2). In addition to the lower stability of uricase-PEG_{Rh}, this PK difference may also be attributed to the larger size of uricase-PEG_{Mw}, allowing it to overcome renal clearance more efficiently.

Notably, uricase-POEGMA outperformed both uricase-PEG_{Rh} and uricase-PEG_{Mw} by extending uricase's circulation by ˜21-fold. Given the head-to-head comparison with both Rₕ- and M_{w}-matched conjugates, we concluded that the superior PK profile was not due to the physical properties of the conjugates known to affect their renal clearance. We previously attributed the superior PK profile of the subcutaneously (s.c.) injected exendin-POEGMA conjugates to POEGMA's amphiphilic structure, allowing slower absorption than the PEG conjugates, thereby delayed elimination. Although this conclusion could still be valid for the s.c. injected drug-POEGMA conjugates, it would not explain the PK profile of the uricase conjugates, given their i.v. administration directly into the bloodstream that allows bypassing the drug absorption phase. Finally, we attributed the superior PK profile of uricase-POEGMA to POEGMA's brush architecture. Together, these results indicated that uricase-POEGMA has a more favorable PK than the PEG conjugates.

Table 2: Summary of PK parameters. PK parameters were derived from the data given in FIG. 3 using non-compartmental analysis. Data showed the mean±SEM. *Derived from the curve fit.

| | | | T_{1/2 elimination} | AUC |
|---|---|---|---|---|
| Mice | Treatment | Cₒ (nM)* | (h) | (nM × h) |
| sNaïve | Uricase | 47.3 ± 1.7 | 2.2 ± 0.1 | 190 ± 16 |
| Mice (1^{st} | Uricase- | 47.4 ± 2.0 | 46.2 ± 1.3 | 2825 ± 121 |
| injection) | POEGMA | | | |
| | Uricase-PEG_{Rh} | 37.1 ± 0.4 | 13.1 ± 0.5 | 1231 ± 80 |
| | Uricase-PEG_{Mw} | 41.3 ± 1.9 | 35.5 ± 1.0 | 1991 ± 153 |
| Immunized | Uricase | 43.0 ± 0.5 | 2.9 ± 0.2 | 219 ± 29 |
| Mice (5^{th} | Uricase- | 47.5 ± 1.6 | 49.6 ± 2.0 | 3064 ± 193 |
| injection) | POEGMA | | | |
| | Uricase-PEG_{Mw} | 34.1 ± 1.1 | 29.6 ± 0.1 | 1623 ± 160 |

### Example 5

### Immunogenicity

Having shown PK benefits of uricase-POEGMA, we next investigated its immunogenicity. This experiment was motivated by the high titers of PEG antibodies induced towards pegloticase treatment that had led to the withdrawal of a PEGylated uricase from the European market. The PEG-specific immune response was presumably due to the cross-linking of the B cell receptors (BCR) by PEG's highly repetitive structure. We hypothesized, without being bound by a particular theory, that uricase-POEGMA conjugates would solve the PEG immunogenicity limitation of uricase-PEG conjugates but were unsure if it would induce anti-POEGMA antibodies due to its significantly higher POEGMA density (˜27 POEGMA vs. 1 per drug), which could lead to BCR cross-linking.

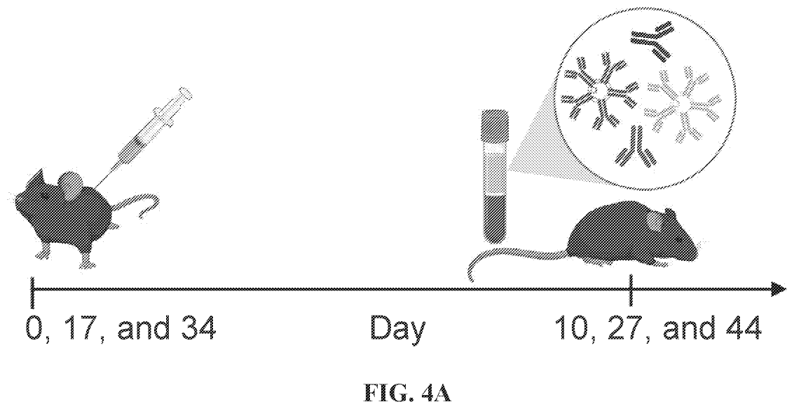

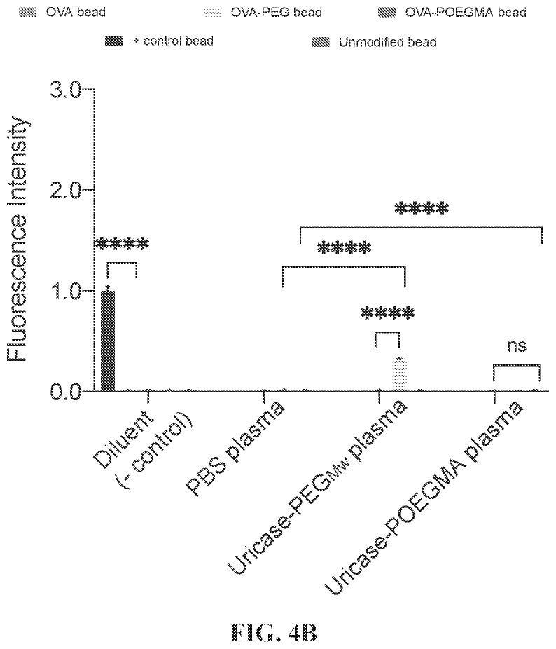

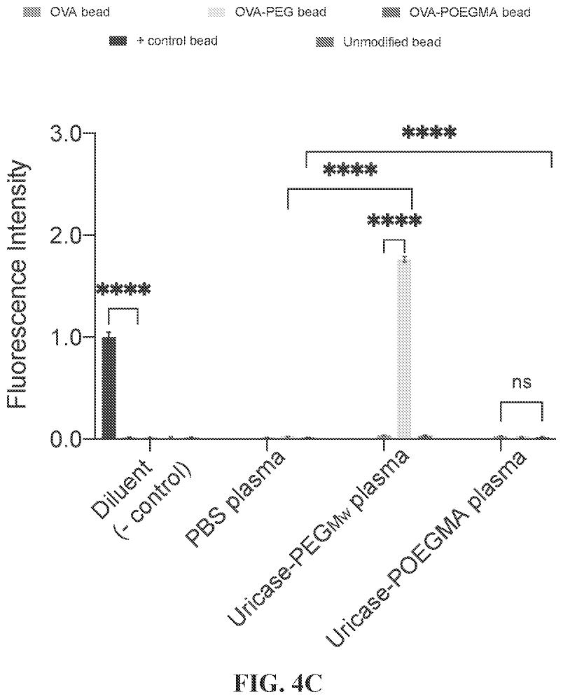

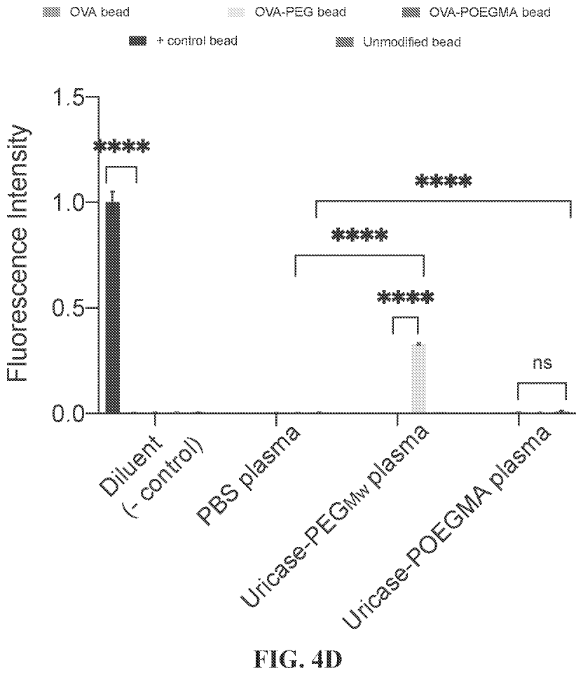

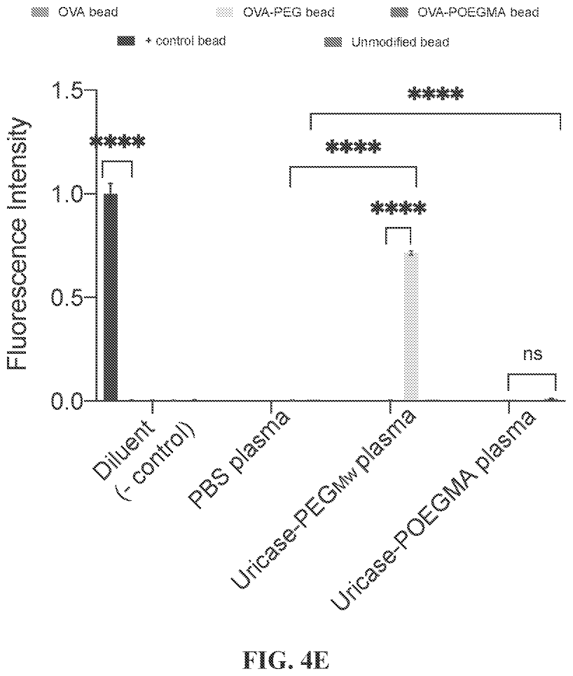

We repeatedly administered sterile and endotoxin-free PBS, uricase-PEG_{Mw}, and uricase-POEGMA into 6-week-old naïve C57BL/6J mice at a dose of 36 nmol kg⁻¹, followed by blood collection and processing into plasma (see the dosing and blood collection regimen in FIG. 4A). We selected the s.c. injection route because the conjugates were exposed to the lymphatic system during absorption into the blood, revealing their immunogenic potential better. This regimen was applied because it allowed us to eliminate the interference of circulating drug on the subsequent immunoassays, indicated by total drug elimination by Day 6 (FIG. 3) while characterizing the development of the ADA response over time.

We used a validated Luminex multiplexed immunoassay. Briefly, the assay uses drug-coupled and fluorescently-barcoded magnetic beads to assess the subtype and specificity of the ADA response. The specificity was tested using an unrelated protein—namely ovalbumin (OVA)—and its PEG and POEGMA conjugates for bead coupling, yielding OVA-, OVA-PEG-, and OVA-POEGMA-coupled beads. If a signal was detected towards these beads in uricase-PEG_{Mw}- or uricase-POEGMA-treated mice plasma, it would indicate PEG- or POEGMA-specific antibodies, respectively. The OVA-coupled bead set was used as a control for cross-reactivity between uricase and OVA. The PBS-treated mice plasma was used as the negative control. Mouse IgM- and IgG-coupled beads incubated in the assay diluent were used as positive assay controls, while the unmodified and drug-coupled beads incubated in the assay diluent were used as negative assay controls.

The assay positive control beads incubated in the assay diluent resulted in high signal, while unmodified and drug-coupled beads had minimal signals (FIG. 4B). No significant background signal was detected, indicated by the lack of signal derived from the plasma samples of mice treated with PBS (FIG. 4B-E). We confirmed the lack of PEG- or POEGMA-specific pre-existing antibodies by testing pre-treatment plasma samples. The OVA-coupled bead had a minimal signal in uricase-PEG_{Mw}- and uricase-POEGMA-treated mice plasma, indicating a lack of cross-reactivity (FIG. 4B-E). Uricase-PEG_{Mw} induced IgM-class PEG-specific antibodies by Day 10, indicated by the significant ADA binding to OVA-PEG-coupled beads in uricase-PEG_{Mw}-treated mice plasma (FIG. 4B). The PEG-specific ADA response persisted until Day 44 (FIG. 4C), increasing with the increasing number of injections. This persistent IgM response indicated a T-cell independent immune response. Uricase-PEG_{Mw} also resulted in a strong IgG-class PEG-specific antibody response (FIG. 4D-E), and the titer increased with each injection, indicating a class-switched PEG-specific antibody response. We previously showed that administering stoichiometric PEG conjugates of highly immunogenic OVA with and without Freund's adjuvant into mice resulted in a strictly IgM-class anti-PEG antibody response. Therefore, we hypothesize, without being bound by a particular theory, that this strong and IgG-class PEG-specific immune response is likely due to the extensive PEGylation of uricase, allowing it to receive T-cell help to mount an IgG-class-switched immune response. Strikingly, uricase-POEGMA induced no IgM or IgG-class anti-POEGMA antibody response (FIG. 4B-E), indicated by the lack of signal detected by the OVA-POEGMA bead. These findings showed that POEGMA remained non-immunogenic even when presented to the immune system at extremely high densities on an immunogenic protein.

### Example 6

### The Effect of Induced PEG Antibodies on PK

We next investigated if induced PEG antibodies affected the PK of the circulating drug. We repeatedly administered uricase-PEG_{Mw} and uricase-POEGMA into, followed by tracking plasma drug concentrations and comparing the first and fifth injection PK data. We also repeatedly administered a separate cohort of mice with uricase to use as a control for specificity. If no change in uricase PK were observed upon repeated exposure while uricase-PEG_{Mw} showed accelerated clearance, it would indicate a PEG antibody-mediated drug elimination.

PK of the uricase and uricase-POEGMA treatments did not change with the repeated injections (Table 2), indicating that neither uricase nor POEGMA induced PK-altering antibodies upon treatment, corroborating with our ADA assessments. Uricase-PEG_{Mw} had significantly shorter t_{1/2 elimination} (35.5 vs. 29.6 h) upon repeated exposure (Table 2). We attributed this altered PK profile to the presence of PK-altering PEG antibodies.

### Example 7

### PEG Antigenicity

Having shown that PEG antibodies led to the early clearance of uricase-PEG_{Mw}, we next investigated if uricase-POEGMA had any reactivity to PEG antibodies. We were unsure that uricase-POEGMA would not show antigenicity to the PEG antibodies. This is because the disclosed conjugate has a much higher POEGMA stoichiometry than previously tested conjugates (˜27 POEGMA chains per drug vs. 1). The repetitive arrangement of POEGMA antigens on the uricase surface could alter epitope exposure to PEG antibodies, thereby result in reactivity to PEG antibodies.

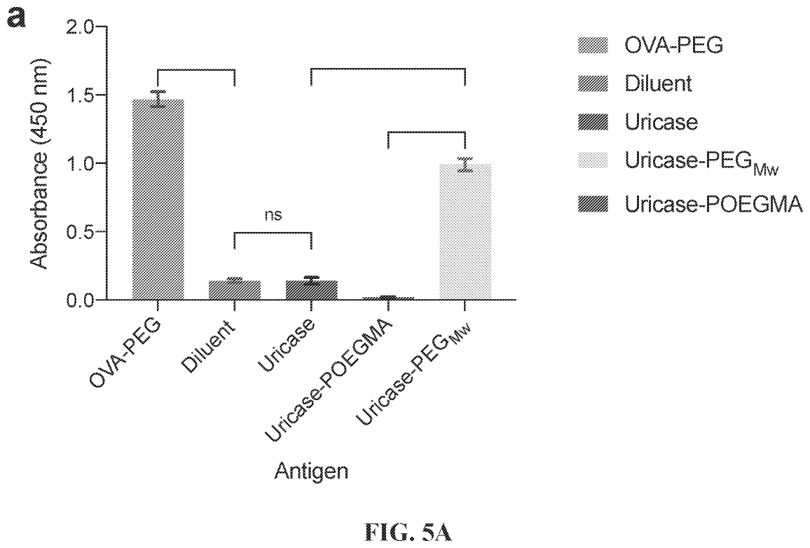

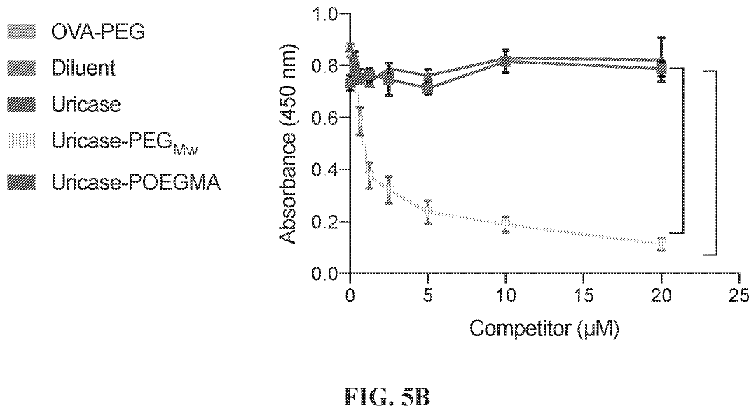

We tested the PEG antigenicity of uricase, uricase-PEG_{Mw}, and uricase-POEGMA using indirect (FIG. 5A) and competitive (FIG. 5B) ELISA. In indirect ELISA, we absorbed the treatments onto a 96-well-plate surface such that the wells had equal amounts of PEG/POEGMA and uricase, followed by probing their reactivity to the PEG antibodies present in an OVA-PEG immunized mice plasma. Diluent and OVA-PEG were used as negative and positive controls, respectively. OVA-PEG had a significant signal, while diluent resulted in only a minimal background (FIG. 5A). Uricase-PEG_{Mw} had a significantly high signal, indicating that the conjugate reacted with the PEG antibodies present in the plasma sample. However, uricase had no significant absorbance because it lacked PEG. Notably, uricase-POEGMA showed no reactivity to the PEG antibodies, suggesting that it was not antigenic to PEG antibodies even when repetitively presented on the drug surface.

The competitive ELISA confirmed the indirect ELISA results. The wells were coated with exendin-PEG that competed with the uricase variants at various concentrations for binding to PEG antibodies. Consistent with the indirect ELISA results, uricase-PEG_{Mw} successfully competed with exendin-PEG, indicated by the significantly lower absorbance at increasing uricase-PEG_{Mw} concentrations (FIG. 5B). However, uricase did not compete with exendin-PEG for binding to PEG antibodies because it does not comprise PEG. Notably, uricase-POEGMA did not bind to PEG antibodies, confirming that PEG antigenicity was eliminated by even extensively POEGMA conjugated drugs.

It is understood that the foregoing detailed description and accompanying examples are merely illustrative and are not to be taken as limitations upon the scope of the invention.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:

Clause 1. A conjugate comprising: a biologically active agent; and a plurality of poly[oligo(ethylene glycol) methyl ether methacrylate] (POEGMA) molecules conjugated to the biologically active agent, each POEGMA molecule having a poly(methyl methacrylate) backbone and a plurality of side chains covalently attached to the backbone, each side chain comprising 2 to 9 monomers of ethylene glycol (EG) repeated in tandem, wherein the conjugate comprises about 5 to about 130 POEGMA molecules per biologically active agent.

Clause 2. The conjugate of clause 1, wherein the conjugate has a reduced immune response relative to a polyethylene glycol (PEG)-biologically active agent conjugate having about 5 to about 130 PEG molecules per biologically active agent.

Clause 3. The conjugate of clause 1 or 2, wherein the conjugate does not induce an anti-POEGMA antibody response.

Clause 4. The conjugate of any one of clauses 1-3, wherein each POEGMA molecule independently has a weight average molecular weight of about 1,000 Da to about 100,000 Da.

Clause 5. The conjugate of any one of clauses 1-4, wherein the conjugate comprises about 25 to about 30 POEGMA molecules per biologically active agent.

Clause 6. The conjugate of any one of clauses 1-5, wherein each side chain comprises 2 to 4 monomers of EG repeated in tandem.

Clause 7. The conjugate of any one of clauses 1-6, wherein the biologically active agent comprises uricase.

Clause 8. The conjugate of any one of clauses 1-7, wherein the biologically active agent is conjugated to the backbone of each POEGMA molecule.

Clause 9. The conjugate of any one of clauses 1-8, wherein the biologically active agent is conjugated to each POEGMA molecule individually through a urethane bond.

Clause 10. The conjugate of any one of clauses 1-9, wherein each side chain has a first terminal end and a second terminal end, wherein the first terminal end is covalently attached to the backbone and the second terminal end comprises an alkyl, ester, amine, amide, or carboxyl group.

Clause 11. A method of reducing the immunogenicity of a polymer-biologically active agent conjugate, the method comprising: conjugating about 5 to about 130 poly[oligo(ethylene glycol) methyl ether methacrylate] (POEGMA) molecules to a biologically active agent, each POEGMA molecule having a poly(methyl methacrylate) backbone and a plurality of side chains covalently attached to the backbone, each side chain comprising 2 to 9 monomers of ethylene glycol (EG) repeated in tandem to provide a conjugate, wherein the conjugate has a reduced immune response relative to a polyethylene glycol (PEG)-biologically active agent conjugate having about 5 to about 130 PEG molecules per biologically active agent.

Clause 12. The method of clause 11, wherein the conjugate does not induce an anti-POEGMA antibody response.

Clause 13. The method of clause 11 or 12, wherein each POEGMA molecule is functionalized with a hydroxyl group, carboxyl group, carbonate group, amine group, ester group, azide group, alkyne group, or a combination thereof prior to conjugating to the biologically active agent.

Clause 14. The method of any one of clauses 11-13, wherein the biologically active agent is conjugated to the backbone of each POEGMA molecule.

Clause 15. The method of any one of clauses 11-14, wherein the biologically active agent is conjugated to each POEGMA molecule individually through a urethane bond.

Clause 16. The method of any one of clauses 11-15, wherein each POEGMA molecule is individually conjugated to the biologically active agent in a non-site-specific manner.

Clause 17. The method of any one of clauses 11-16, wherein each POEGMA molecule independently has a weight average molecular weight of about 1,000 Da to about 100,000 Da.

Clause 18. The method of any one of clauses 11-17, wherein the conjugate comprises about 25 to about 30 POEGMA molecules per biologically active agent.

Clause 19. The method of any one of clauses 11-18, wherein each side chain comprises 2 to 4 monomers of EG repeated in tandem.

Clause 20. The method of any one of clauses 11-19, wherein each side chain has a first terminal end and a second terminal end, wherein the first terminal end is covalently attached to the backbone and the second terminal end comprises an alkyl, ester, amine, amide, or carboxyl group.

What is claimed is:

1. A conjugate comprising:
a biologically active agent; and
a plurality of poly[oligo(ethylene glycol) methyl ether methacrylate] (POEGMA) molecules conjugated to the biologically active agent, each POEGMA molecule having a poly(methyl methacrylate) backbone and a plurality of side chains covalently attached to the backbone, each side chain comprising 2 to 9 monomers of ethylene glycol (EG) repeated in tandem,
wherein the conjugate comprises about 5 to about 130 POEGMA molecules per biologically active agent.

2. The conjugate of claim 1, wherein the conjugate has a reduced immune response relative to a polyethylene glycol (PEG)-biologically active agent conjugate having about 5 to about 130 PEG molecules per biologically active agent.

3. The conjugate of claim 1, wherein the conjugate does not induce an anti-POEGMA antibody response.

4. The conjugate of claim 1, wherein each POEGMA molecule independently has a weight average molecular weight of about 1,000 Da to about 100,000 Da.

5. The conjugate of claim 1, wherein the conjugate comprises about 25 to about 30 POEGMA molecules per biologically active agent.

6. The conjugate of claim 1, wherein each side chain comprises 2 to 4 monomers of EG repeated in tandem.

7. The conjugate of claim 1, wherein the biologically active agent comprises uricase.

8. The conjugate of claim 1, wherein the biologically active agent is conjugated to the backbone of each POEGMA molecule.

9. The conjugate of claim 1, wherein the biologically active agent is conjugated to each POEGMA molecule individually through a urethane bond.

10. The conjugate of claim 1, wherein each side chain has a first terminal end and a second terminal end, wherein the first terminal end is covalently attached to the backbone and the second terminal end comprises an alkyl, ester, amine, amide, or carboxyl group.

11. A method of reducing the immunogenicity of a polymer-biologically active agent conjugate, the method comprising:
conjugating about 5 to about 130 poly[oligo(ethylene glycol) methyl ether methacrylate] (POEGMA) molecules to a biologically active agent, each POEGMA molecule having a poly(methyl methacrylate) backbone and a plurality of side chains covalently attached to the backbone, each side chain comprising 2 to 9 monomers of ethylene glycol (EG) repeated in tandem to provide a conjugate,
wherein the conjugate has a reduced immune response relative to a polyethylene glycol (PEG)-biologically active agent conjugate having about 5 to about 130 PEG molecules per biologically active agent.

12. The method of claim 11, wherein the conjugate does not induce an anti-POEGMA antibody response.

13. The method of claim 11, wherein each POEGMA molecule is functionalized with a hydroxyl group, carboxyl group, carbonate group, amine group, ester group, azide group, alkyne group, or a combination thereof prior to conjugating to the biologically active agent.

14. The method of claim 11, wherein the biologically active agent is conjugated to the backbone of each POEGMA molecule.

15. The method of claim 11, wherein the biologically active agent is conjugated to each POEGMA molecule individually through a urethane bond.

16. The method of claim 11, wherein each POEGMA molecule is individually conjugated to the biologically active agent in a non-site-specific manner.

17. The method of claim 11, wherein each POEGMA molecule independently has a weight average molecular weight of about 1,000 Da to about 100,000 Da.

18. The method of claim 11, wherein the conjugate comprises about 25 to about 30 POEGMA molecules per biologically active agent.

19. The method of claim 11, wherein each side chain comprises 2 to 4 monomers of EG repeated in tandem.

20. The method of claim 11, wherein each side chain has a first terminal end and a second terminal end, wherein the first terminal end is covalently attached to the backbone and the second terminal end comprises an alkyl, ester, amine, amide, or carboxyl group.
